(19) United States
(12) Patent Application Publication
(10) Pub. No.: US 2023/0009211 A1
(43) Pub. Date: Jan. 12, 2023

(54) SWELLABLE POLYMER HYBRID FIBRES FOR A SLEEVE OF AN INTRALUMINAL ENDOPROSTHESIS

(71) Applicant: CORTRONIK GMBH, Rostock (DE)

(72) Inventors: Stefanie Kohse, Rostock (DE); Kerstin Lebahn, Rostock (DE); Niels Grabow, Rostock (DE); Dalibor Bajer, Rostock (DE); Swen Grossmann, Rostock (DE); Klaus-Peter Schmitz, Rostock (DE); Heinz Mueller, Rostock (DE); Carsten Momma, Rostock (DE); Sabine Illner, Rostock (DE); Daniela Arbeiter, Rostock (DE); Thomas Eickner, Rostock (DE)

(21) Appl. No.: 17/780,144

(22) PCT Filed: Nov. 24, 2020
(86) PCT No.: PCT/EP2020/083169

§ 371 Date: May 26, 2022

(30) Foreign Application Priority Data
Dec. 4, 2019 (DE) 10 2019 132 970.1

(51) Int. Cl. A61L 31/02; A61L 31/10; A61L 31/14; C08L 67/04; A61L 31/16

(52) U.S. Cl. CPC A61L 31/022; A61L 31/10; A61L 31/145; C08L 67/04; A61L 31/16

(57) ABSTRACT

An intraluminal endoprosthesis such as a stent has a metallic supporting structure and a sleeve surrounding the supporting structure. The sleeve can include fibres which are applied to an outer side of the supporting structure. The fibres each have a polymer core and a hydrogel casing connected thereto. The sleeve can also be formed from a fibre mixture of polymer and hydrogel fibres.

### PRIORITY CLAIM

This application is a 35 U.S.C. 371 US National Phase and claims priority under 35 U.S.C. § 119, 35 U.S.C. 365(b) and all applicable statutes and treaties from prior PCT Application PCT/EP2020/083169, which was filed Nov. 24, 2020, which application claimed priority from German Application Ser. No. 10 2019 132 970.1, which was filed Dec. 4, 2019.

### FIELD OF THE INVENTION

A field of the invention is intraluminal endoprostheses, including stents (for example a coronary stent or a peripheral stent).

### BACKGROUND

Over the past 20 years, the number of interventional vascular (coronary and peripheral) interventions has increased continuously. At the same time, increasingly more complex lesions that are more difficult to access are being treated on the one hand, and the number of older patients and patients with poor vessel condition (for example calcified and brittle vessels) is increasing on the other hand. This has meant that, with an increasingly greater number of patients, the vessels are being damaged during surgery by the catheters and guide wires used.

Perforations or ruptures of the treated vessels may occur as a result. Perforations or ruptures are, in essence, especially in the case of coronary vessels, very serious, life-threatening complications, and therefore must be treated immediately.

In the coronary area, for example, various so-called stent grafts are available for such treatment. The currently available implants consist of a permanent main body made of a metal (generally Co—Cr alloys) and a permanent polymer sleeve, preferably made of PTFE or a polyurethane, which seals the damage in the vessel wall. This sleeve may be a simple polymer tube or a tissue that is secured to the stent lying therebeneath or thereabove.

Since a rupture or perforation of the vessel of the above-described kind is an immediately life-threatening complication, the handling of such systems during use is a particularly key aspect. Here, it is of utmost importance that the systems can be handled easily and quickly and can be supplied to the implantation site. It is also important that the rupture at the implantation site is sealed as quickly and reliably as possible.

The most frequently described clinical complications in this case are as follows:
loss or displacement of the endoprosthesis (for example stent) from the balloon of the catheter system used for implantation,
failure to reach the cell lesion, passage through constricted points,
a defective seal,
acute and subacute in-stent thromboses.

Depending, inter alia, on the size of the perforation in the vessel wall, in the majority of cases in which such endoprostheses or stent grafts are used, there is a further disadvantage that once the vessel wall has healed to such an extent (approximately 2-5 days) that no more blood can escape through the perforated or ruptured point (haemostasis), the implants then no longer have any function to perform. Subsequently, however, the permanent polymer sleeves (polymer tubes or spun sleeves) in particular pose a problem, since they permit an endothelialisation of the inner vessel side only very slowly and incompletely and thus generally significantly increase the risk of thrombosis or the risk of a vessel occlusion. In addition, the normal vascular peristalsis is suppressed by the generally very rigid implants. The main problems or complications are caused, for the above-mentioned reasons, by the permanent polymer sleeve; the permanent supporting structure therebeneath poses only a much smaller problem.

### SUMMARY OF THE INVENTION

An intraluminal endoprosthesis includes a supporting structure including or consisting of one of the following materials: stainless steel, Co-based alloy, Ni-based alloy, Ni—Ti alloy, Pt-containing Fe—Cr—Ni alloy, Ti—, Nb— or Ta-based alloys. A sleeve surrounds the supporting structure. The sleeve can include fibres that each have a polymer core and a hydrogel casing connected thereto. The sleeve can be formed from a fibre mixture of polymer fibres and hydrogel fibres.

### BRIEF DESCRIPTION OF THE DRAWINGS

Embodiments and also further features and advantages of the invention will be explained hereinafter with reference to the drawings, in which:

FIG. 1 shows a schematic depiction of an embodiment of the method according to the invention and of an endoprosthesis according to the invention produced by the method; and

FIG. 2 shows an image of an electrospun polyionic liquid (PIL) taken by scanning electron microscopy;

FIG. 3 shows the swelling behaviour of a biocompatible hybrid hydrogel formed from poly(vinylisopropylimidazolium bromide)/poly(N-isopropylacrylamide) (ratio 30/70 wt. %) (abb.:PILl/pNIPAAm), shown via a shaped body (cylinder); and

FIG. 4 shows a schematic depiction of an embodiment of an alternative method according to the invention and of an endoprosthesis according to the invention produced by said method.

### DETAILED DESCRIPTION OF THE PREFERRED EMBODIMENTS

An improved intraluminal endoprosthesis is provided that avoids an excessively large profile of the system formed of catheter and endoprosthesis (for example stent), and an excessively low flexibility of the system formed of catheter and stent graft. Furthermore, an excessive permeability of the sleeve of the endoprosthesis and an excessive layer thickness of the sleeve is avoided. Enhanced mechanical load-bearing capacity of the sleeve is provided by the expansion of a balloon-expandable supporting structure arranged beneath, the adhesion of the endoprosthesis to the balloon catheter, and the visibility under X-ray of the endoprosthesis.

An intraluminal endoprosthesis is disclosed, in particular in the form of a stent or stent graft (for example a coronary stent or a peripheral stent), having a supporting structure and a sleeve surrounding the supporting structure, which sleeve includes fibres which are applied by electrospinning to the outer side of the supporting structure, wherein the fibres each have a polymer core and a hydrogel casing connected thereto, or the sleeve is formed from a mixture of polymer fibres and hydrogel fibres.

Here, the fibres are preferably applied to the outer side of the supporting structure by coaxial electrospinning, wherein a polymer solution and a preliminary hydrogel product or a hydrogel precursor are dispensed simultaneously via a coaxial nozzle, so that a Taylor cone with an inner polymer component and an outer, coaxial hydrogel component is formed and the two substances are dispensed from the nozzle in the form of a thread in a coaxial arrangement. In this case, the outer hydrogel component is preferably produced in such a way that the polymer solution contains the polymer and at least reactive monomeric, oligomeric or polymeric hydrogel precursors (uncrosslinked hydrogel), whereas a suitable substance for crosslinking the precursor(s) to the hydrogel is guided in the outer component. The desired hydrogel casing (sleeve) thus forms externally around the polymer core. Alternatively, the outer hydrogel component is produced in such a way that the inner polymer solution contains the support polymer and a suitable substance for crosslinking the hydrogel precursor, which is then supplied in the outer component. What is key in both embodiments is that the suitable substance for crosslinking and the hydrogel precursor only come into contact and react with each other once they have exited the coaxial nozzle. Correspondingly, it is also possible to encase an inner polymer solution with a hydrogel precursor by electrospinning and to produce the crosslinking to form the hydrogel post-process (after the electrospinning process), by applying a suitable substance for crosslinking subsequently. A hydrogel precursor, within the scope of this application, is understood to be any conceivable substance, in particular a reactive monomeric, oligomeric or polymeric precursor (uncrosslinked), that forms a hydrogel after coming into contact with a suitable substance for crosslinking. Within the scope of this application, a hydrogel is understood to be a crosslinked biomacromolecule that absorbs large amounts of water without dissolving. In other words, a hydrogel within the scope of this invention does not dissolve spontaneously upon contact with a water-containing bodily fluid (for example blood), but instead degrades in a degradation process or not at all.

Alternatively, the sleeve is preferably applied by dual electrospinning to the outer side of the supporting structure. Here, the polymer and hydrogel fibres are produced at the same time by parallel electrospinning (two nozzles or more) and form a composite material. The hydrogel fibre may be produced here by mixing a hydrogel precursor with a suitable substance for crosslinking, wherein the hydrogel precursor is contacted with the suitable substance for crosslinking just before, during or after their dispensing from the nozzle.

The crosslinking to form a hydrogel may be performed both as a chemical crosslinking and as a physical crosslinking, by changing certain ambient conditions, such as temperature, ultraviolet light, ion concentration, or pH value.

All natural and synthetic bi- and multi-functional compounds (crosslinkers), which for example originated from the group of: dialdehydes, carbodiimides, diamines, diazirines, diacrylates, diisocyanates, bisacrylamides, preferably genipin, methylene bisacrylamide, glutaraldehyde, succinimide derivatives and hexamethylene diamine, are suitable as substances for the chemical crosslinking of the hydrogel precursors.

All biogenic electrolytes, preferably with ions of sodium, potassium, calcium, magnesium, chloride, phosphate and hydrogen carbonate may be used as suitable substances for the ionic crosslinking.

Furthermore, organic salts may be added to the solvent in order to produce polyelectrolyte fibres. When generating polymerised and crosslinked ionic liquids (PILs), ionic liquids, in which the melting point is less than 100° C., may be used initially as hydrogel precursors. These contain alkylated cationic compounds, for example from the group: imidazolium, pyridinium, pyrrolidinium, guanidinium, uronium, thiouronium, piperidinium, morpholinium, ammonium and phosphonium and anionic pendants, for example from the group: halides, tetrafluoroborates, trifluoroacetates, triflates, hexafluorophosphates, phosphinates and tosylates. Pharmaceutically active ionic liquids, which usually contain as anions an FDA-authorised medicament, which is generally used in a clinical setting in the form of (sodium) salts, are preferably contained. Examples include: diclofenac, acetylsalicylic acid, sulfamethoxazole, sulfadiazine, chloramphenicol, fosfomycin, dalbavancin, rifampicin, minocycline and others.

If the crosslinking is based on the use of ultraviolet light, ionic or radical, preferably biocompatible type-1 and type-2 photoinitiators may be used in addition (0.01-5 wt. %). Suitable photoinitiators in this include, for example, α-ketoester-based photoinitiators, but also all α-hydroxy-, α-alkoxy- or α-amino-aryl ketones or also acylphosphine oxides, for example 2-hydroxy-2-methyl-1-phenyl-propan-2-one, 2-hydroxy-1-[4-(2-hydroxy ethoxy)-phenyl]-2-methylpropan-1-one, 2,2-dimethoxy-2-phenylacetophenone, 1-[4-(2-hydroxyethoxy)-phenyl]-2-hydroxy-2-methyl-1-propan-1-one, 1-hydroxycyclohexyl phenyl ketone or trimethylbenzoyldiphenylphosphine oxide.

The present invention is based on that fact that, by the hydrogel component, in particular the permeability of the sleeve is reduced and an adhesive effect between endoprosthesis and a balloon catheter system possibly used for implantation is improved.

Supporting structures (in particular stents), and also the sleeves arranged thereon, are currently divided into two basic types: the permanent or long-term supporting structures or sleeves and biodegradable supporting structures or sleeves. Permanent supporting structures or sleeves are designed so that they may remain in the vessel or at the implantation site in the human or animal body for an unspecific period of time. By contrast, biodegradable supporting structures or sleeves are broken down in the vessel or body over a predetermined period of time. Biodegradable supporting structures are preferably broken down only once the traumatized tissue of the vessel has healed and therefore the supporting structure no longer needs to remain in the vessel lumen or body. Furthermore, a biodegradable sleeve is preferably broken down only when it no longer needs to provide a sealing effect.

The supporting structure may be a self-expanding supporting structure or a balloon-expandable supporting structure. For a balloon-expandable supporting structure, production from a tube is possible, in particular, which tube is cut, for example with the aid of a laser. In the case of a self-expanding supporting structure, this may be formed for example from a suitable wire.

The supporting structure is preferably mesh-like and is formed by interconnecting bars, which delimit openings in the supporting structure. The bars or openings may be formed for example by the laser cutting from a tube. A supporting structure manufactured from a wire may also have a mesh-like structure.

In accordance with one embodiment of the intraluminal endoprosthesis, it is provided that the supporting structure of the endoprosthesis includes one of the following materials or is formed from one of the following materials: stainless steel, Co-based alloy, in particular Co—Cr alloys such as L605, Ni-based alloy, Ni—Ti alloy, in particular nitinol, Pt-containing Fe—Cr—Ni alloy, Ti—, Nb— or Ta-based alloys.

In accordance with an alternative embodiment of the intraluminal endoprosthesis, it is provided that the supporting structure of the endoprosthesis includes one of the following materials or is formed from one of the following materials: an Mg alloy; an Mg—Al—Zn alloy; an Mg—Al—Mn alloy; an Mg—Al—Zn—Mn alloy; an Mg-RE alloy, wherein RE is selected from the group of rare earths; an Mg—Y-RE alloy, wherein RE is selected from the group of rare earths; an Mg-RE-Zn alloy, wherein RE is selected from the group of rare earths; an Mg—Al—Y alloy; an Mg—Al-RE alloy, wherein RE is selected from the group of rare earths; an Mg—Zn—Zr alloy, an Mg—Ca—Zn alloy; an Mg—Al alloy with an Al content of from 3 wt. % to 11 wt. %; an Mg—Ca—Zn alloy with a Zn content of from 0.01 wt. % to 12 wt. %, preferably of from 0.1 wt. % to 5 wt. %, and a Ca content of from 0.01 wt. % to 5 wt. %, preferably of from 0.1 wt. % to 1 wt. %; an Mg—Y-RE alloy, wherein RE stands for further rare earths different from Y, with a Y content of from 0.1 wt. % to 5 wt. %, an Nd content of from 0.01 wt. % to 5 wt. %, a Gd content of from 0.01 wt. % to 3 wt. %, a Dy content of from 0.01 wt. % to 3 wt. %, and wherein the alloy optionally includes 0.1 wt. % to 1 wt. % Zr and other rare earths.

Furthermore, in accordance with one embodiment of the intraluminal endoprosthesis it is provided that the polymer core or the polymer fibre includes at least one biodegradable polymer which is selected from the group consisting of: polylactide, poly-L-lactide; poly-D,L-lactide; poly-L-lactide-co-D,L-lactide; polyglycolide; polyanhydride; polyhydroxybutyrate; polyhydroxyvalerate; poly-ε-caprolactone; polydioxanone; poly(lactide-co-glycolide); poly(lactide-co-caprolactone); poly(ethyleneglycol-co-caprolactone); poly(glycolide-co-caprolactone); poly(hydroxybutyrate-co-valerate); polytrimethylene carbonate-based polymer; polypropylene succinate.

In accordance with one embodiment of the invention, the at least one degradable polymer may be a copolymer which includes two or more different monomers of the polymers from the aforementioned group.

Furthermore, the at least one degradable polymer in accordance with one embodiment of the invention may be present in a mixture or a blend, wherein the mixture includes two or more different polymers of the above-mentioned group. Here, a blend is a macroscopically homogeneous mixture of two or more different polymers.

Furthermore, in accordance with one embodiment of the invention, the at least one biodegradable polymer of the polymer solution is preferably one of the following substances: polyhydroxybutyrate; a copolymer including hydroxybutyrate; polyvalerate; a copolymer including valerate.

In accordance with one embodiment of the intraluminal endoprosthesis it is also provided that the polymer is a poly-D,L-lactide-co-glycolide, with a lactide proportion of from 5 wt. % to 85 wt. %, preferably with a lactide proportion of from 50 wt. % to 85 wt. %.

In an alternative embodiment of the invention it is provided that the polymer core or the polymer fibre includes at least one polymer which is selected from the group consisting of: polyurethanes; polyamides; polysulfones; poly siloxanes; polymethylmethacrylates; styrene-butadiene block copolymers; polyimides; poly carbonates; polyureas; polyethylene oxides; polyvinylpyrrolidones; polyglycolic acids and copolymers, mixtures or blends thereof.

In accordance with one embodiment of the intraluminal endoprosthesis it is also provided that the hydrogel casing or the hydrogel fibre includes at least one biodegradable hydrogel selected from the group consisting of: polysaccharide; hyaluronic acid (crosslinked); cellulose (modified); chitosan; alginate; pectinate; agarose; agar; casein; chitosan alginate; gelatine; dextran; dextran-dialdehyde gelatine (crosslinked); proteins; collagen.

The particular biodegradable hydrogel may also be present in original or derivatised form. Furthermore, the biodegradable hydrogel may be a mixture of the aforementioned hydrogels.

In an alternative embodiment of the invention it is provided that the hydrogel casing (sleeve) or the hydrogel fibre includes at least one hydrogel which is selected from the group consisting of: polyelectrolytes, in particular polymerisable vinylogous ionic liquids (PILs); pharmaceutical ionic liquids; polyvinylpolypyrrolidone (PVPP); polyacrylates (PA); polyvinyl acrylates (PVA); polyacrylamides (PAA), in particular poly(N-isopropylacrylamide) (pNIPAAm); poly oxazolines (POZ); polyvinyl ethers; polyphosphazenes in original or derivatised form, and mixtures thereof as linearly polymerised or crosslinked hydrophilic networks.

Furthermore, in accordance with one embodiment of the intraluminal endoprosthesis, it is provided that an active substance (in particular a medicament) is incorporated into the polymer core or the polymer fibre or is anchored to the surface thereof, wherein the active substance is selected from the group consisting of: an active substance (in particular a medicament) which assists endothelialisation; an active substance with anti-proliferative effect; an active substance with anti-inflammatory effect; an active substance with antithrombotic effect; an active substance including ECM macromolecules; collagen; elastin; laminine; fibronectin; a cell-binding protein, in particular RGD; a growth factor, in particular VEGF or PDEC; sirolimus; paclitaxel; everolimus; mycophenolic acid; angiopeptin; enoxaparin; hirudin; acetylsalicylic acid; dexamethasone; rifampicin; minocycline; budesonide; desonide; corticosterone; cortisone; hydrocortisone; prednisolone; heparin; a heparin derivative; urokinase; PPACK.

Furthermore, in accordance with one embodiment of the intraluminal endoprosthesis it is provided that an active substance (in particular a medicament) represents either a component of the hydrogel precursor or is incorporated in the hydrogel casing or is anchored to the surface thereof, wherein the active substance is selected from the group consisting of: an active substance with coagulation-promoting effect; fibrinogen; calcium; thrombin, thrombokinase; an antifibrinolytic; para-aminomethylbenzoic acid; tranexamic acid; aprotinin; chelate; citrate; EDTA; protamine; vitamin K; a wound-healing or tissue-like substance for promoting the formation of new tissue and/or cell integration and/or cell attachment; a stimulating factor; a growth factor; a substance having its own cells; a substance having keratinocytes; fibrin fibres, an extracellular matrix protein; collagen; laminine; hyalurone; an active substance with antithrombotic effect; an active substance having ECM macromolecules; collagen; elastin; laminine; fibronectin; a cell-binding protein, in particular RGD; a growth factor, in particular VEGF or PDEC; sirolimus; paclitaxel; everolimus; mycophenolic acid; angiopeptin; enoxaparin; hirudin; acetylsalicylic acid; dexamethasone; rifampicin; minocycline; budesonide; desonide; corticosterone; cortisone; hydrocortisone; prednisolone; heparin; a heparin derivative; urokinase; PPACK.

Furthermore, in accordance with one embodiment of the intraluminal endoprosthesis it is provided that the supporting structure includes, in addition to the sleeve, a polymer coating which forms a surface of the supporting structure on which the sleeve is arranged, wherein the polymer coating is preferably designed to elute a medicament incorporated therein or a pharmacological active substance.

In accordance with the embodiment of the intraluminal endoprosthesis, it is provided in this regard that the medicament incorporated into the polymer coating is selected from the group consisting of: a medicament with antiproliferative effect; a medicament with anti-inflammatory effect; a medicament with antithrombotic effect; sirolimus; paclitaxel; everolimus; mycophenolic acid; angiopeptin; enoxaparin; hirudin; acetylsalicylic acid; dexamethasone; rifampicin; minocycline; budesonide; desonide; corticosterone; cortisone; hydrocortisone; prednisolone; heparin; a heparin derivative; urokinase; PPACK.

Furthermore, in accordance with one embodiment of the intraluminal endoprosthesis it is provided that the sleeve, in particular the polymer core of the particular fibre, the polymer fibre and/or the polymer coating of the supporting structure includes a substance that is visible under X-ray, selected from the group consisting of: a zircon compound, in particular a pure or a stabilised zircon compound; zircon dioxide; zircon carbide; tantalum; a tantalum compound; barium sulfate; silver; silver iodide; gold; platinum; palladium; iridium; copper; iron oxide; titanium; alkali iodide; an iodised aromatic substance; an iodised aliphate; an iodised oligomer; an iodised polymer.

The substance visible under X-ray may also be formed by a mixture that includes two or more of the aforementioned substances, or by an alloy that includes two or more of the aforementioned metals.

Furthermore, in accordance with one embodiment of the intraluminal endoprosthesis, it is provided that the hydrogel casing of the particular fibre or the hydrogel fibre has an adhesive property so that the sleeve forms an adhesion to a balloon of a balloon catheter when the endoprosthesis is arranged on the balloon and the hydrogel casing of the fibres or the hydrogel fibres of the sleeve contact the balloon.

By such an additional adhesion to the balloon material, the holding force of the endoprosthesis on the balloon is increased. Since the fibres of the sleeve have an adhesive effect in relation to the balloon material, a further optimisation of the usage properties may be achieved. With the usual assembly processes, balloon folds are also embedded during the crimping process in order to improve the adhesion of the stent on the balloon.

In accordance with one embodiment of the invention, a slight adhesion to these balloon folds is formed by the selection of an adhesive polymer or hydrogel additive for the hydrogel of the particular hydrogel casing or the hydrogel fibre, for example polyvinylpyrrolidone (PVP) or polyethylene glycol (PEG), whereby the holding force of the endoprosthesis on the balloon may be considerably increased. During the expansion of the balloon, this adhesion connection is detached by the stretching of the balloon sleeve and the balloon may be removed without difficulty.

The adhesion of the sleeve to the balloon folds may be achieved in particular by:
1) post-processing of multimodal polymer systems (UV crosslinking/curing; thermal or chemical treatment by, for example, temperature change or etching processes)
2) during the crimping (mechanical/thermal adhesion/gluing to the individual crimp points)
3) thermal post-treatment (for example by heating the balloon to temperatures˜ 50° C.)
4) embedding of the endoprosthesis with balloon in agarose gels for example, which solidify spontaneously at 4° C. (applied at the end of the processing chain and kept cool).

In accordance with a further aspect of the invention, an intraluminal endoprosthesis is disclosed, in particular a stent (for example coronary stent or peripheral stent), including a supporting structure and a sleeve surrounding the supporting structure, which sleeve includes polymer fibres which are applied by electrospinning to an outer side of the supporting structure, and wherein the sleeve includes hydrogel fibres which are applied by electrospinning to the outer side of the supporting structure.

Here, the polymer and hydrogel fibres are produced by parallel dual electrospinning of a polymer solution and of a hydrogel precursor, wherein the polymer solution is dispensed via a first nozzle and the hydrogel precursor is dispensed simultaneously via a second nozzle and the two form a composite material. The hydrogel fibre is preferably formed here in such a way that a hydrogel precursor is mixed with a suitable substance for crosslinking shortly before/upon entry into the second nozzle or after exit from the second nozzle. By the dual electrospinning, a sleeve made of a fibre mixture which consists of polymer fibres and hydrogel fibres forms on the supporting structure. The created, preferably biodegradable sleeve is thus a composite material.

The endoprostheses described herein, in accordance with one embodiment, may each have a higher density of the fibres of the sleeve so as to avoid a fraying of the sleeve during the laser cutting of the sleeve edges or the supporting structure.

A further aspect of the present invention relates to a method for producing an intraluminal endoprosthesis according to one of the preceding claims, wherein the method includes the steps of:
providing the metallic supporting structure, and
applying the fibres to the outer side of the supporting structure by dual or coaxial electrospinning of a polymer solution and a hydrogel precursor to generate a composite of polymer fibres and hydrogel fibres or the polymer core and the hydrogel casing of the particular fibre.

During the coaxial electrospinning, the fibres are applied to the outer side of the supporting structure, wherein the polymer solution and the hydrogel precursor are dispensed simultaneously via a coaxial nozzle, so that a Taylor cone including an inner polymer component and an outer, coaxial hydrogel component is formed and the two substances are dispensed in the form of a fibre from the coaxial nozzle, wherein the polymer component forms a polymer core of the fibre and the hydrogel component forms a hydrogel casing surrounding the polymer core. Here, the outer hydrogel component is preferably produced in such a way that the polymer solution contains the polymer and at least one reactive monomeric, oligomeric or polymeric precursor (hydrogel precursor, uncrosslinked), whereas a suitable substance for crosslinking the precursor(s) to form the hydrogel is guided in the outer component. The desired hydrogel sleeve thus forms externally around the polymer core. Alternatively, the outer hydrogel component is produced in such a way that the inner polymer solution contains the support polymer and a suitable substance for crosslinking the hydrogel precursor, which is supplied in the outer component. What is key in both embodiments is that the suitable substance for crosslinking and the hydrogel precursor only come into contact and react with each other once they have exited the coaxial nozzle. Correspondingly, it is also possible to produce an inner polymer solution and a hydrogel precursor as a sleeve by electrospinning and to produce the hydrogel subsequently by the post-process application (after the electrospinning process) of a suitable substance for crosslinking.

A method is also disclosed for producing the intraluminal endoprosthesis according to a further aspect of the invention, the method including the steps of:
providing the metallic structure, and
applying the polymer fibres to the outer side of the supporting structure by dual electrospinning of a polymer solution and simultaneously applying the hydrogel fibres to the outer side of the supporting structure by electrospinning of the hydrogel precursor.

The polymer and hydrogel fibres are thus produced here preferably by dual electrospinning of a polymer solution and a hydrogel precursor, wherein the polymer solution is dispensed via a first nozzle and the hydrogel precursor is dispensed simultaneously via a second nozzle. The hydrogel is preferably formed here in such a way that a reactive monomeric, oligomeric or polymeric precursor (hydrogel precursor, uncrosslinked) is mixed with a suitable substance for crosslinking just before or upon entry into the second nozzle or after the exit from the second nozzle. The second nozzle then dispenses the mixture of hydrogel precursor and crosslinking substance which forms the electrospun fibre on the endoprosthesis which, together with the polymer fibre applied simultaneously by electrospinning to the supporting structure, forms a fibre mixture (composite material).

During the electrospinning the polymer for the core or polymer fibre is dissolved in a suitable organic solvent (mixture). The hydrogel precursor for the casing fibre or hydrogel fibre is preferably dissolved in a water-based solvent, but is not limited to this. Further solvent components may be alcohols, such as methanol, trifluoroethanol or hexafluoroisopropanol; acids such as acetic acid or trifluoroacetic acid; dimethylformamide; tetrahydrofuran; dichloromethane; dimethylsulfoxide and other organic solvents in all possible mixtures and ratios.

With regard to the methods described herein, the above-described polymers may be used for the polymer cores or the polymer fibres. Furthermore, the above-described hydrogels may be used for the hydrogel casings or the hydrogel fibres.

The embodiments described above with a sleeve formed of fibres with a polymer core and a hydrogel sleeve, similarly to the disclosed variant of a sleeve that includes polymer fibres and hydrogel fibres, also has the advantage that the endoprosthesis may assume a very small cross-section when introduced at the implantation site. The hydrogel component of the fibre sleeve or the hydrogel fibres also absorb water from the bodily vessel following implantation in the bodily vessel and swell. It is thus possible to produce a sleeve having the same low permeability as could be achieved without hydrogel component in the fibre sleeve or hydrogel fibres only with a much thicker sleeve. Due to the small cross-section, the insertion of the endoprosthesis is simplified and is less traumatic.

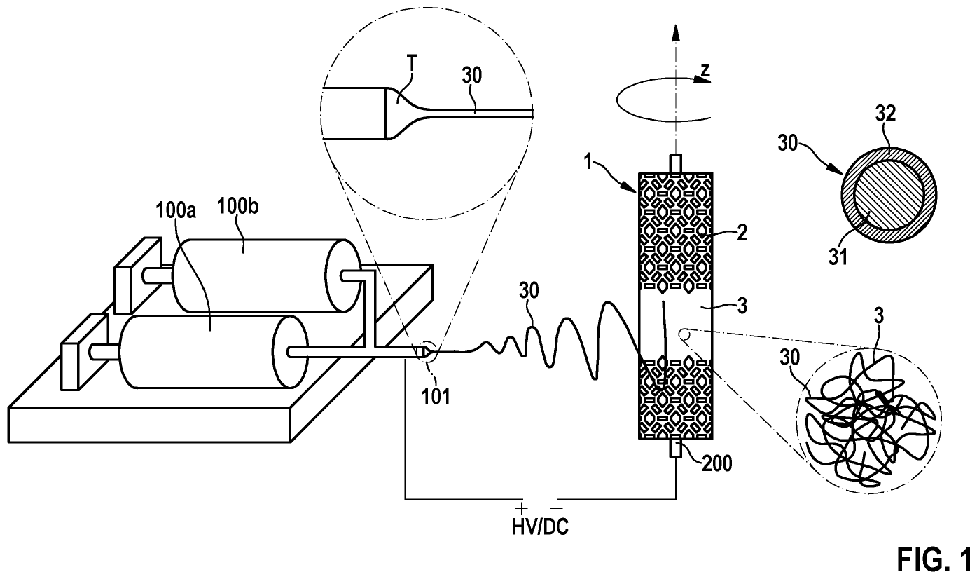

FIG. 1 shows an embodiment of a method according to the invention for producing an intraluminal endoprosthesis 1, in particular in the form of a stent, wherein the endoprosthesis 1 has a supporting structure 2 and a sleeve 3 arranged on the supporting structure 2.

The sleeve 3 is, in particular, a nonwoven sleeve 3 produced by coaxial electrospinning from what are known as core-shell fibres 30, and is spun onto the supporting structure 2.

The fibres 30 forming the sleeve consist here of an inner core fibre 31 in the form of a polymer core 31 and a casing fibre 32 produced thereon in the form of a hydrogel casing 32. The polymer of the core fibre 31 is in this case a polymer disclosed herein and the casing fibre 32 is a (rapidly swellable) hydrogel disclosed herein (see above, for example). The sleeve 3 is applied here to the supporting structure 2 in the non-expanded state of the supporting structure 2. The layer thickness of the sleeve is significantly below the thicknesses of currently commercially available products required until now.

In the coaxial electrospinning according to FIG. 1, the fibres 30 are applied to the outer side of the supporting structure 2 by a coaxial nozzle 101, wherein a polymer solution 10 containing the used polymer and the hydrogel are dispensed simultaneously via the coaxial nozzle, so that a Taylor cone T with an inner polymer component 31 and an outer, coaxial hydrogel component 32 is formed and the two substances are dispensed in the form of a thread from the nozzle 101.

The polymer solution and a hydrogel precursor of the same repeating unit are stored in the store 100a. The store 100b contains a substance for crosslinking the hydrogel precursor. The polymer store 100a is connected to an inner nozzle opening and the store 100b with the substance for crosslinking is connected to a coaxial, outer nozzle opening of the coaxial nozzle.

An electrical voltage is applied between the nozzle 101, which is also referred to as an emitter, and a collector 200, on which the supporting structure 2 is arranged, and for example may lie in the range of from 4 kV to 8 kV. The supporting structure 2 is arranged on the collector 200 and may be rotated (for example by the collector 200) about a longitudinal axis and in particular moved along the longitudinal axis z, in order to distribute the polymer and hydrogel components in fibre form on the outer side of the supporting structure 2. The substance for crosslinking the hydrogel precursor crosslinks the latter after exit from the nozzle 101 and after contact with the supporting structure 2, so that the polymer core 31 of the fibre 30 forms a hydrogel sleeve 32.

Due to the particular morphology of the sleeve or the hybrid nonwoven 3 produced in this way, there is a pronounced peripheral expansion of the hybrid fibres by swelling immediately after implantation of the outer hydrogen component 32 upon contact with aqueous media. This leads advantageously to a rapid sealing of the sleeve 3.

Due to the use of these hybrid fibres 30, it is therefore possible to produce very thin sleeves 3 with a high porosity, which, upon contact with blood, develop the necessary sealing effect with low permeability. An advantage of the significantly reduced layer thickness of the sleeve 3 according to the invention is, in particular, a reduced profile of the endoprosthesis 1 in the crimped state at the time of insertion at the implantation site, which promotes a simple and gentle implantation of the endoprosthesis 1.

A further advantage of the hybrid fibres 30 lies in the possibility of producing a dual-drug depot, which enables an acute release of an active substance from the hydrogel casing 32 and a delayed release of an (other) active substance from the polymer core fibre. The polymer core fibre is illustrated by 31.

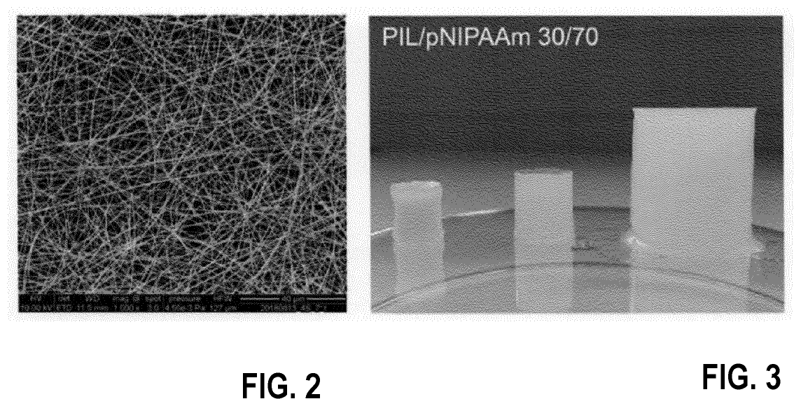

FIG. 2 shows, for explanation purposes, an image of a electrospun polyionic liquid (PIL, also referred to as polyelectrolyte) taken by scanning electron microscopy, and FIG. 3 shows the above-mentioned significant swelling behaviour of a biocompatible hybrid hydrogel formed from poly(vinylisopropylimidazolium bromide)/poly(N-isopropylacrylamide) (ratio 30/70 wt. %) (abb.:PILl/pNIPAAm).

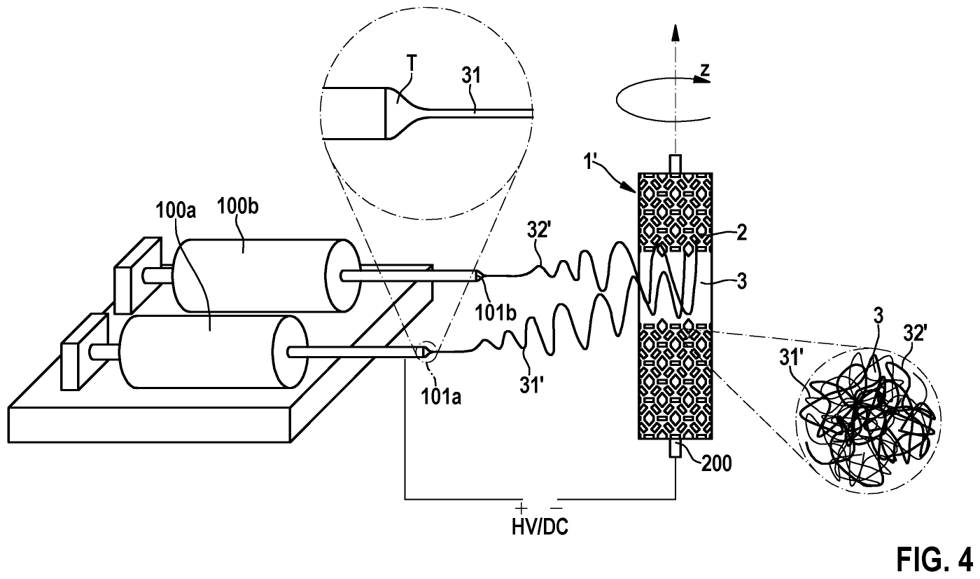

A further aspect of the invention relates to an endoprosthesis 1′ according to FIG. 4, having a biodegradable sleeve 3 in the form of a nonwoven sleeve 3 formed from a hybrid material. Here, a biodegradable supporting structure 2 is covered by a nonwoven structure 3 formed from a hybrid material via parallel dual electrospinning (see FIG. 4). The hybrid fibre material is a multi-component fibre system formed from individual fibres of two components. The polymer of the first fibres 31′ is, in this case, a biodegradable polymer disclosed herein and the (separate) second fibres 32′ are a (rapidly swellable) hydrogel disclosed herein. In the parallel electrospinning, the substances (polymer and hydrogel) are dispensed via two separate nozzles 101a, 101b, wherein an electrical voltage is again applied between the particular nozzle 101a, 101b and the collector 200, on which the supporting structure 2 is arranged, which voltage, at the particular nozzle 101a, 101b, allows the formation of a Taylor cone T for the thread or fibre formation of the particular material (polymer or hydrogel respectively). The supporting structure 2 is rotatable or movable in the above-described manner in order to facilitate a uniform formation of the sleeve 3. The second fibres 32′ are formed here in such a way that a substance for crosslinking to form a hydrogel is added (not shown) to the hydrogel precursor before the nozzle 101b. The substance for crosslinking the hydrogel precursor crosslinks this after exit from the nozzle 101b and after contact with the supporting structure 2, so that a hydrogel fibre 32′ forms.

The covering of the supporting structure 2 with the sleeve 3 or the fibres 31′, 32′ is also performed in the non-expanded state of the supporting structure 2. The layer thickness of the sleeve 3 also lies here significantly below the previously required thicknesses of currently commercially available products.

Due to the particular morphology of the hybrid nonwoven 3 formed from the individual fibres 31′, 32′, there is a pronounced peripheral expansion of the hydrogel fibres 32′ by swelling immediately after implantation upon contact with aqueous media. This leads to a rapid sealing of the entire sleeve 3. Due to the use of this multi-component fibre system 31′, 32′, it is possible to produce very thin sleeves 3 of high porosity, which, upon contact with blood, develop the necessary sealing effect with low permeability. The advantage of the significantly reduced layer thickness of the sleeve 3 is in particular a reduced profile of the endoprosthesis 1′ in the crimped state, which promoted a simple and gentle implantation of the endoprosthesis. Here as well, a further potential advantage of this multi-component fibre system lies in the possibility to produce a dual-drug depot, which allows an acute release of an active substance from the hydrogel component/fibre 32′ and a delayed release of an (other) active substance from the polymer component/fibre 31′.

The present invention allows the avoidance of the majority of the potentially serious complications of previous approaches by:
a thinnest possible profile of the catheter tip resulting from the reduced sleeve thickness, which makes it possible to pass even through narrow vessel passages without difficulty, especially also since pre-dilations or the like in the available time are often very problematic,
a most flexible possible system of endoprosthesis 1, 1′ and used catheter, so that the catheter may also pass through complex vessel passages with low force expenditure,
a firmest possible adhesion of the endoprosthesis 1, 1′ to the balloon of the catheter used, in order to minimise the risk that the endoprosthesis 1, 1′ might be stripped off when passing through narrow vessel portions and under the necessary quick working conditions,
a best possible visibility of the implant, so that a quickest and most accurate placement possible may be provided, and
a best and quickest possible sealing of the rupture or perforation, so that the damaged point of the vessel may be closed as quickly as possible.

These improvements ensure that, in the emergency situation, which is always present when using such implants, the point to be sealed in the vessel is reached as quickly as possible and the implant may be implanted as securely as possible. Here, it must be noted in particular that the wall thickness of the supporting structure 2 plus the sleeve 3 regularly makes up for more than 25% of the crossing profile of the overall system, and at the same time the sleeve 3 itself may have approximately twice the wall thickness of the supporting structure.

The invention thus allows the use of a much thinner sleeve 3 (lower layer thickness) with simultaneous sufficient sealing by the generation of hydrogel-like fibre structures (water-insoluble) from permanent or degradable polymer materials, which allow a rapid diffusion of water into the polymer matrix, absorb this, retain it, and swell with a significant increase in volume, without loss of their cohesion in the network. The required fibre density of the electrospun sleeve for sealing may be significantly reduced by the swelling capacity of the fibre structures in the event of contact with water.

By a reduction of the thickness of the particular sleeve 3, but also by a reduction of the wall thickness of the supporting structure 2, the usage properties of the implant 1, 1′ may be significantly improved.

A further improvement may be achieved in that such a thinner sleeve 3 has a lower rigidity and the system as a whole is thus more flexible. A further optimisation of the usage properties may be achieved if the fibres 30, 31′, 32′ of the particular sleeve 3 with implantation properties have the ability to swell with the surrounding blood and thus allow a particularly reliable sealing of the rupture or perforation.

A further possible optimisation of the usage properties may be achieved if the fibres 30, 31′, 32′ of the particular sleeve 3 have an adhesive effect in relation to the balloon material. With the usual assembly processes, balloon folds are also embedded during the crimping process in order to improve the adhesion of the endoprosthesis 1, 1′ (for example stent) on the balloon. If a suitable sleeve 3 now has a slight adhesion to these balloon folds, the retaining force of the endoprosthesis 1, 1′ on the balloon may thus significantly increase. As the balloon is expanded, this adhesion connection is detached by the stretching of the balloon sleeve, and the balloon may be removed without difficulty.

Part of the present invention thus relates to a sleeve 3 having particular properties which are intended to make it possible to achieve the best possible applicability of the implant 1, 1′ in the above-mentioned sense. Due to the use of the coaxial electrospinning to produce an endoprosthesis 1, the spectrum of usable polymers is extended to hydrophilic polymeric nonwoven structures. Due to the use of hydrophilic polymers, the permeability of blood into the endoprosthesis 1 is promoted, and at the same time a passage of liquid is prevented by swelling of the fibres 30 (increase in volume of the hydrogel casing 32). A key advantage of the use of biocompatible hydrogels lies in the acceleration of the ingrowth behaviour, since the cells penetrate the hydrophilic polymer network of the hydrogel component 32 and may use this as a support structure. Complete ingrowth of the sleeve 3 is thus advantageously promoted.

Due to the arrangement of hybridisation of the various polymeric fibre structures (caused by the production process) on the supporting structure 2, it is possible to coordinate the degradation over time of the sleeve 3 and supporting structure 2.

With hybridisation with degradable, swellable fibres, the cell colonisation is further increased by reduction of the fibre density, whereby the ingrowth behaviour is promoted. The addition of active substances into the biodegradable hydrophilic nonwoven structures allows a time-resolved (simultaneous, acute and delayed) active substance release both for immediately supporting the treatment of the vessel rupture and also for the ongoing improvement of the ingrowth or breakdown behaviour of the endoprosthesis.

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

1. An intraluminal endoprosthesis comprising: a supporting structure and a sleeve surrounding the supporting structure, wherein the sleeve comprises fibres applied to the outer side of the supporting structure, wherein
the fibres each have a polymer core and a hydrogel casing connected thereto; or
the sleeve is formed from a fibre mixture of polymer fibres and hydrogel fibres.

2. The intraluminal endoprosthesis according to claim 1, wherein the supporting structure comprises or consists of one of the following materials or is formed from one of the following materials: stainless steel, Co-based alloy, a Co—Cr alloy, an Ni-based alloy, an Ni—Ti alloy, nitinol, Pt-containing Fe—Cr—Ni alloy, Ti—, Nb— or Ta-based alloys.

3. The intraluminal endoprosthesis according to claim 2, wherein the polymer core or the polymer fibre comprises at least one biodegradable polymer which is selected from the group consisting of: polylactide; poly-L-lactide; poly-D,L-lactide; poly-L-lactide-co-D,L-lactide; polyglycolide; poly-D,L-lactide-co-glycolide; polyanhydride; polyhydroxybutyrate; polyhydroxyvalerate; poly-ε-caprolactone; polydioxanone; poly(lactide-co-glycolide); poly(lactide-co-caprolactone); poly(ethyleneglycol-co-caprolactone); poly(glycolide-co-caprolactone); poly(hydroxybutyrate-co-valerate); polytrimethylene carbonate-based polymer; polypropylene succinate.

4. The intraluminal endoprosthesis according to claim 3, wherein the at least one biodegradable polymer is a poly-D,L-lactide-co-glycolide, with a lactide proportion of from 5 wt. % to 85 wt. %.

5. The intraluminal endoprosthesis according to claim 2, wherein the polymer core or the polymer fibre comprises at least one polymer which is selected from the group consisting of: polyurethanes; polyamides; polysulfones; polysiloxane s; polymethylmethacrylates; styrene-butadiene block copolymers; polyimides; polycarbonates; polyureas; polyethylene oxides; polyvinylpyrrolidones; polyglycolic acids and copolymers, mixtures or blends thereof.

6. The intraluminal endoprosthesis according to claim 1, wherein the hydrogel casing or the hydrogel fibre comprises at least one biodegradable hydrogel which is selected from the group consisting of: polysaccharide; hyaluronic acid (crosslinked); cellulose (modified); chitosan; alginate; pectin; agarose; agar; casein; chitosan alginate; gelatine; dextran; dextran-dialdehyde gelatine (crosslinked); proteins; collagen.

7. The intraluminal endoprosthesis according to claim 1, wherein the hydrogel casing or the hydrogel fibre comprises at least one hydrogel, which is selected from the group consisting of: polyelectrolytes, in particular polymerisable vinylogous ionic liquids (PILs); pharmaceutical ionic liquids; polyvinylpolypyrrolidone (PVPP); polyacrylates (PA); polyvinyl acrylates (PVA); polyacrylamides (PAA), in particular poly(N-isopropylacrylamide) (pNIPAAm); polyoxazolines (POZ); polyvinyl ethers; polyphosphazenes in original or derivatised form, and mixtures thereof as linearly polymerised or crosslinked hydrophilic networks.

8. The intraluminal endoprosthesis according to claim 1, comprising an active substance is incorporated into or anchored to a surface of the polymer core or the polymer fibre, wherein the active substance is selected from the group consisting of: an active substance which assists endothelialisation; an active substance with anti-proliferative effect; an active substance with anti-inflammatory effect; an active substance with antithrombotic effect; an active substance comprising ECM macromolecules; collagen; elastin; laminine; fibronectin; a cell-binding protein, in particular RGD; a growth factor, in particular VEGF or PDEC; sirolimus; paclitaxel; everolimus; mycophenolic acid; angiopeptin; enoxaparin; hirudin; acetylsalicylic acid; dexamethasone; rifampicin; minocycline; budesonide; desonide; corticosterone; cortisone; hydrocortisone; prednisolone; heparin; a heparin derivative; urokinase; PPACK.

9. The intraluminal endoprosthesis according to claim 1, comprising an active substance is incorporated into or anchored to a surface of the hydrogel casing or the hydrogel fibre, wherein the active substance is selected from the group consisting of: an active substance with coagulation-promoting effect; fibrinogen; calcium; thrombin, thrombokinase; an antifibrinolytic; para-aminomethylbenzoic acid; tranexamic acid; aprotinin; chelate; citrate; EDTA; protamine; vitamin K; a wound-healing or tissue-like substance for promoting the formation of new tissue and/or cell integration and/or cell attachment; a stimulating factor; a growth factor; a substance having its own cells; a substance having keratinocytes; fibrin fibres, an extracellular matrix protein; collagen; laminine; hyalurone.

10. The intraluminal endoprosthesis according to claim 1, comprising a polymer coating on the supporting structure.

11. The intraluminal endoprosthesis according to claim 10, comprising a medicament incorporated into the polymer coating, wherein the medicament is selected from the group consisting of: a medicament with antiproliferative effect; a medicament with anti-inflammatory effect; a medicament with antithrombotic effect; sirolimus; paclitaxel; everolimus; mycophenolic acid; angiopeptin; enoxaparin; hirudin; acetylsalicylic acid; dexamethasone; rifampicin; minocycline; budesonide; desonide; corticosterone; cortisone; hydrocortisone; prednisolone; heparin; a heparin derivative; urokinase; PPACK.

12. The intraluminal endoprosthesis according to claim 1, wherein the sleeve comprises a substance that is visible under X-ray, selected from the group consisting of: a zircon compound, tantalum; a tantalum compound; barium sulfate; silver; silver iodide; gold; platinum; palladium; iridium; copper; iron oxide; titanium; alkali iodide; an iodised aromatic substance; an iodised aliphate; an iodised oligomer; an iodised polymer.

13. The intraluminal endoprosthesis according to claim 1, wherein the sleeve comprises an adhesive to adhere to a balloon of a balloon catheter when the endoprosthesis is arranged on the balloon.

14. A method for producing an intraluminal endoprosthesis according to claim 1, wherein the method comprises the steps of:
providing the supporting structure, and
applying the fibres to the outer side of the supporting structure by dual or coaxial electrospinning of a polymer solution and a hydrogel precursor to generate a composite of polymer fibres and hydrogel fibres or fibres with the polymer core and the hydrogel casing.

15. The intraluminal endoprosthesis according to claim 1, wherein the supporting structure comprises a Co—Cr alloy.

16. The intraluminal endoprosthesis according to claim 15, wherein the Co—Cr Alloy is L605.

17. The intraluminal endoprosthesis according to claim 1, wherein the supporting structure comprises nitinol.

18. The intraluminal endoprosthesis according to claim 4, wherein the lactide proportion is from 50 wt. % to 85 wt. %.

19. The intraluminal endoprosthesis according to claim 12, wherein the zircon compound comprises a stabilised zircon compound; zircon dioxide, or zircon carbide.

20. The intraluminal endoprosthesis according to claim 10, wherein the polymer coating is a medicament eluting polymer.
